(19) United States
(12) Patent Application Publication
(10) Pub. No.: US 2026/0363692 A1
(43) Pub. Date: Nov. 19, 2026

(54) RETRACTOR SYSTEM

(71) Applicant: Thompson Surgical Instruments, Inc., Traverse City, MI (US)

(72) Inventor: William White, Traverse City, MI (US)

(21) Appl. No.: 19/675,108

(22) Filed: May 12, 2026

Related U.S. Application Data
(60) Provisional application No. 63/805,078, filed on May 13, 2025.

(51) Int. Cl. A61B 17/02

(52) U.S. Cl. CPC A61B 17/0293

(57) ABSTRACT

The present disclosure relates generally to a surgical retractor system. The surgical retractor system may include a carriage and a frame that passes laterally through the carriage. The carriage may include a carriage body, an arm clamp, a frame channel, a retaining clip, and a knob. The arm clamp may be coupled to the carriage body and configured to secure a retractor arm. The frame channel may pass laterally through the carriage body and permit passage of the frame through the carriage body. The retaining clip may include a detent that prevents removal of the frame from the frame channel when in a retaining position. The knob may include a thread shaft that passes through a lateral side of the carriage body. Tightening of the knob may force the frame clamp piston toward the surface of the frame.

### RELATED APPLICATIONS

This application claims benefit from U.S. Patent Application No. 63/805,078, filed May 13, 2025, the disclosure of which is hereby incorporated by reference in its entirety.

### FIELD

The present disclosure relates to a surgical apparatus and, in particular, to a retractor system used to retract soft tissue about a surgical incision or other operative site.

### BACKGROUND

The present disclosure relates to retractor systems and retractors that are used to retract soft tissue and other anatomy of a patient.

During a surgical procedure, a surgeon may make an incision in a patient to access internal organs, bones, and/or other anatomical structures. Retractors may be used to hold back soft tissue and other patient anatomy in the immediate area of the incision. Such retractors may provide the surgeon with a view of the internal organs, bones, and/or other anatomical structures. Furthermore, the retractors may maintain an opening via which the surgeon may access the anatomical structures with one or more surgical tools.

The usefulness of various retractor devices is limited by the number of ways that the retractor may be positioned with respect to the retracted anatomy as well as the ease with which the surgeon may adjust the relative position of the retractor both before and during surgery. The less obstructive, the less obtrusive, and the more versatile a retractor device, the more desirable it becomes for use in the above-described manner. Further limitations and disadvantages of conventional and traditional approaches will become apparent to one of skill in the art, through comparison of such approaches with the present disclosure as set forth in the remainder of the present application with reference to the drawings.

### SUMMARY

Various aspects of this disclosure are related to a surgical retractor system that includes retractors used to retract anatomy in order to provide exposure of an operative site. In some embodiments, the surgical retractor system may include a carriage and a frame that passes laterally through the carriage. The carriage may include a carriage body, an arm clamp, a frame channel, a retaining clip, and a knob. The arm clamp may be coupled to the carriage body and configured to secure a retractor arm. The frame channel may pass laterally through the carriage body and permit passage of the frame through the carriage body. The retaining clip may include a detent that prevents removal of the frame from the frame channel when in a retaining position. The knob may include a thread shaft that passes through a lateral side of the carriage body. Tightening of the knob may force the frame clamp piston toward the surface of the frame, thus securing the carriage body to the frame. Tightening of the knob may further tighten the arm clamp around the retractor arm.

### BRIEF DESCRIPTION OF THE DRAWINGS

Various features and advantages of the present disclosure may be more readily understood with reference to the following detailed description taken in conjunction with the accompanying drawings, wherein like reference numerals designate like structural elements.

FIG. 1 depicts an example retractor system in accordance with various aspects of the present disclosure.

FIG. 2 depicts a first perspective view of an example carriage of the retractor system shown in FIG. 1.

FIG. 3 depicts a second perspective view of the example carriage of the retractor system shown in FIG. 1.

FIG. 4 depicts a translucent view of the example carriage shown in FIGS. 2 and 3.

FIG. 5 depicts a cross-section view of the example carriage shown in FIGS. 2 and 3.

### DETAILED DESCRIPTION

Various aspects of the present disclosure may be embodied in many different forms and should not be construed as being limited to the example embodiments set forth herein. Rather, these example embodiments of the disclosure are provided so that this disclosure will be thorough and complete and will convey various aspects of the disclosure to those skilled in the art.

The following discussion presents various aspects of the present disclosure by providing examples thereof. Such examples are non-limiting, and thus the scope of various aspects of the present disclosure should not necessarily be limited by any particular characteristics of the provided examples. In the following discussion, the phrases “for example,” “e.g.,” and “exemplary” are non-limiting and are generally synonymous with “by way of example and not limitation,” “for example and not limitation,” and the like. The various aspects and embodiments disclosed herein are not intended to be limiting. Other embodiments may be utilized, and other changes may be made, without departing from the spirit or scope of the subject matter presented herein. It will be readily understood that the aspects of the present disclosure, as generally described herein and illustrated in the figures, may be arranged, substituted, combined, separated, and designed in a wide variety of different configurations, all of which are contemplated herein.

As utilized herein, “or” means any one or more of the items in the list joined by “or.” As an example, “x or y” means any element of the three-element set {(x), (y), (x, y)}. In other words, “x or y” means “one or both of x and y.” As another example, “x, y, or z” means any element of the seven-element set {(x), (y), (z), (x, y), (x, z), (y, z), (x, y, z)}. In other words, “x, y, or z” means “one or more of x, y, and z.”

The terminology used herein is for the purpose of describing particular examples only and is not intended to be limiting of the disclosure. As used herein, the singular forms are intended to include the plural forms as well, unless the context clearly indicates otherwise. It will be further understood that the terms “comprises,” “includes,” “comprising,” “including,” “has,” “have,” “having,” and the like when used in this specification, specify the presence of stated features, integers, steps, operations, elements, and/or components, but do not preclude the presence or addition of one or more other features, integers, steps, operations, elements, components, and/or groups thereof.

It will be understood that, although the terms first, second, etc. may be used herein to describe various elements, these elements should not be limited by these terms. These terms are only used to distinguish one element from another element. Thus, for example, a first element, a first component, or a first section discussed below could be termed a second element, a second component, or a second section without departing from the teachings of the present disclosure. Similarly, various spatial terms, such as “upper,” “lower,” “side,” and the like, may be used in distinguishing one element from another element in a relative manner. It should be understood, however, that components may be oriented in different manners, for example a device may be turned sideways so that its “top” surface is facing horizontally and its “side” surface is facing vertically, without departing from the teachings of the present disclosure.

In the drawings, various dimensions may be exaggerated for illustrative clarity. Additionally, like reference numbers are utilized to refer to like elements through the discussions of various examples.

Unless specified otherwise, the term “coupled” may be used to describe two elements directly contacting each other or describe two elements indirectly connected by one or more other elements. For example, if element A is coupled to element B, then element A may be directly contacting element B or indirectly connected to element B by an intervening element C. Similarly, the terms “over” or “on” may be used to describe two elements directly contacting each other or describe two elements indirectly connected by one or more other elements.

Also, any numerical range recited herein is intended to include all sub-ranges subsumed therein. For example, a range of “1 to 10” is intended to include any and all sub-ranges between and including the recited minimum value of 1 and the recited maximum value of 10, that is, all sub-ranges beginning with a minimum value equal to or greater than 1 and ending with a maximum value equal to or less than 10, and all sub-ranges in-between, e.g., 1 to 6.3, or 5.5 to 10, or 2.7 to 6.1.

The discussion will now refer to various example illustrations provided to enhance the understanding of the various aspects of the present disclosure. It should be understood that the scope of this disclosure is not limited by the specific characteristics of the examples provided and discussed herein.

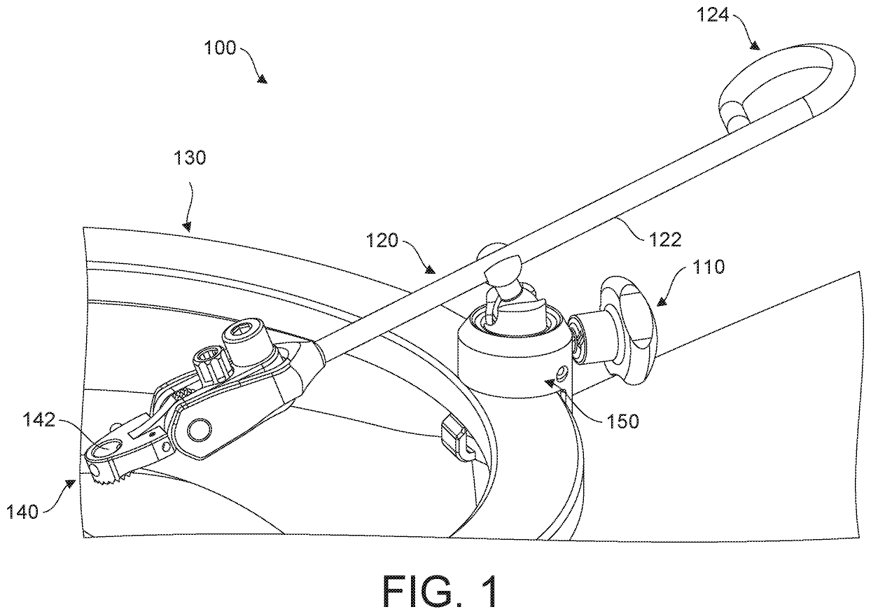

FIG. 1 illustrates an embodiment of a retractor system 100 in accordance with various aspects of the present disclosure. The retractor system 100 may include a carriage 110, a retractor arm 120, a frame 130, and a retractor connector 140.

The retractor arm 120 may include one or more longitudinal arm members 122 between a proximal end of the retractor arm 120 and a distal end of the retractor arm 120. In the depicted embodiment, the retractor arm 120 may comprise a single longitudinal arm member 122 having a circular cross section. However, other embodiments may include additional longitudinal arm members 122 and/or longitudinal arm members 122 with different cross sections (e.g., square, rectangular, oval, etc.). As explained in greater detail below, the carriage 110 may engage and secure the retractor arm 120 to the frame 130 through its engagement with one or more exterior surfaces of a longitudinal arm member 122.

The distal end of the retractor arm 120 may include a retractor handle 124. In some embodiments, the retractor handle 124 may have a circular shape as shown. However, in other embodiments, the retractor handle 124 may have various other shapes such as semi-circular, curved, or a straight continuation of the retractor arm 120.

In some embodiments, the retractor arm 120 may include various adjustable joints that permit articulation of the retractor arm 120 and positioning of the retractor connector 140 with respect to the carriage 110 and the frame 130. For example, the retractor arm 120 may include one or more universal joints, ball joints, prismatic joints, etc. located along the length of the retractor arm 120 and between respective longitudinal arm members 122. In various embodiments, each longitudinal arm member 122 may be a generally straight, generally cylindrical member. However, in some embodiments, one or more of the longitudinal arm members 122 may be bent, curved, angled, etc. in one or more locations to accommodate different procedures or access sites.

The frame 130 may include a top side, a bottom side, and one or more lateral sides (e.g., an inner lateral side and an outer lateral side) between the top side and the bottom side. In some embodiments, the frame 130 may be formed in various ways and in various shapes. While the present disclosure contemplates the frame 130 as a circular shape, the frame 130 may be various other shapes such as, for example, an oval, a rectangle with rounded corners, a square with rounded corners, etc. Similarly, while the present disclosure contemplates the frame 130 as being continuous and permitting the carriage 110 to move freely throughout the entire length of the frame 130, the frame 130 may be non-continuous in some embodiments.

The retractor connector 140 may include a top side, a bottom side, and one or more lateral sides between the top side and the bottom side. In particular, the retractor connector 140 may include a left lateral side, a right lateral side, a proximal lateral side, and a distal lateral side opposite the proximal lateral side. The proximal lateral side of the retractor connector 140 may be coupled to the distal end of the retractor arm 120. Moreover, the retractor connector 140 may include an attachment port 142 that passes vertically through the top side and bottom side of the retractor connector 140. The attachment port 142 may be sized to receive and secure an attachment post of a retractor blade. Though such attachment to the retractor connector 140, a retractor blade may be secured to the frame 130 via the retractor arm 120.

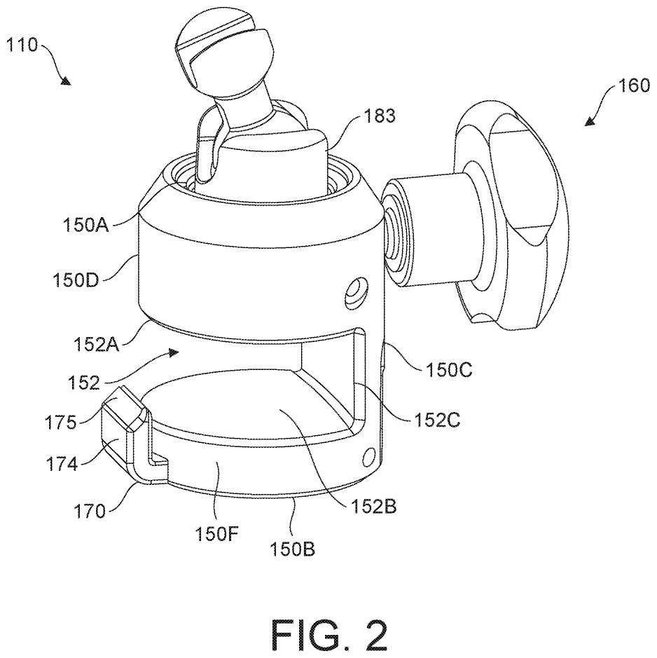

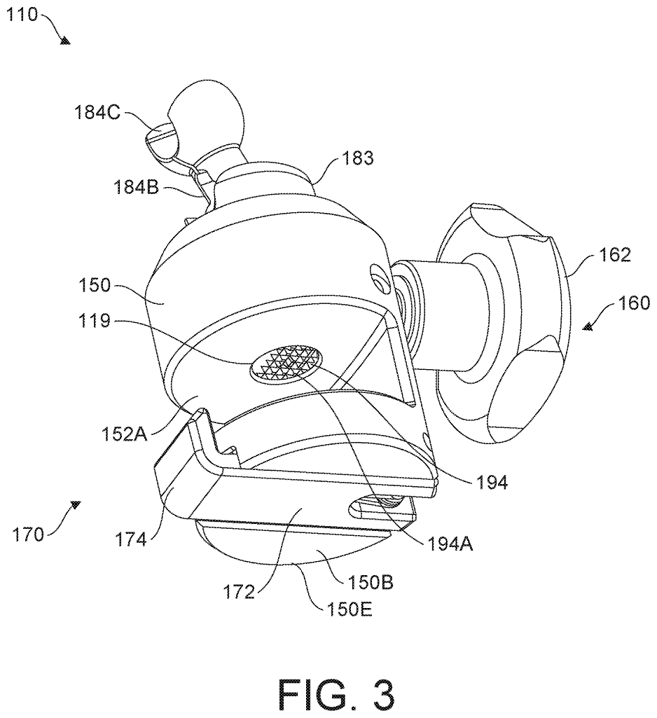

FIGS. 2 and 3 provide perspective views of the carriage 110 of the retractor system 100 shown in FIG. 1. In general, the carriage 110 may include a carriage body 150, a control device or knob 160, a retaining clip 170, an arm clamp 180, and a frame clamp 190.

The carriage body 150 may include a top side 150A, a bottom side 150B, a back side 150C, a front side 150D, a left side 150E, and a right side 150F. The carriage body 150 may further include a frame channel 152. The frame channel 152 may extend through the front side 150D toward the back side 150C and may pass laterally through the carriage body 150 from the left side 150E to the right side 150F. The frame channel 152 may be sized to receive and permit passage of the frame 130 laterally through the carriage body 150. To this end, the frame channel 152 may comprise a top surface 152A, a bottom surface 152B, and a back surface 152C that engage and/or closely mate with corresponding external surfaces or sides of the frame 130 when received.

In various embodiments, the top surface 152A and the bottom surface 152B may be implemented as planar surfaces that run parallel to one another so as to engage and guide the top side and the bottom side of the frame 130 when received in frame channel 152. In some embodiments, the top surface 152A and the bottom surface 152B of the frame channel 152 may be various shapes corresponding to a cross section or partial cross section of the carriage body 150. The present disclosure contemplates a carriage body 150 having a generally circular cross section, thus resulting in surfaces 152A, 152B having a generally a semi-circular shape. However, in some embodiments, the shape of the carriage body 150 and/or the surfaces 152A, 152B may have other shapes and corresponding lateral cross sections (e.g., circular, elliptical, square, rectangular, hexagonal, polygonal, etc.).

The control device or knob 160 may include a handle 162 and a threaded shaft 164. The threaded shaft 164 may include a proximal end coupled to the handle 162 and a distal end threaded through a threaded opening in the carriage body 150. In the depicted embodiment, the threaded shaft 164 may pass through a threaded opening in the back side 150C of the carriage body 150. However, in other embodiments, the threaded shaft 164 of the knob 160 may pass through a threaded opening of a different side of the carriage body 150.

Rotation of the knob 160 (e.g., via manual rotation of its handle 162) in a tightening direction may decrease a distance between the handle 162 and the back side 150C of the carriage body 150. Such tightening may also advance a distal end or tip of the threaded shaft 164 toward a central portion of the carriage body 150. Conversely, rotation of the knob 160 in a loosening direction may increase a distance between the handle 162 and the back side 150C of the carriage body 150. Such loosening may also withdrawal a distal end or tip of the threaded shaft 164 from the central portion of the carriage body 150. While depicted with a generally circular cross section, the handle 162 of the knob 160 may be implemented using various other shapes and/or configurations.

As shown, the retaining clip 170 may be positioned in a predetermined notch or recess in the bottom side 150B of the carriage body 150. The retaining clip 170 may comprise a longitudinal member 172 having a proximal end pivotally coupled to the carriage body 150 toward the back side 150C of the carriage body 150 and a distal end that extends beyond the front side 150D of the carriage body 150. The retaining clip 170 further includes a retaining clip detent 174 at its distal end. When in a retaining or locked position, the retaining clip detent 174 may extend upwardly and beyond the bottom surface 152B of the frame channel 152. In this manner, the retaining clip detent 174 may block, obstruct, and/or otherwise prevent extraction of the frame 130 from the frame channel 152 when the retaining clip 170 is in the close or locked position. Furthermore, a front side 175 of the retaining clip detent 174 may be tapered to aid the loading of the frame 130 into the frame channel 152. In particular, an outer lateral side of the frame 130 may engage the tapered front side 175 of the retaining clip detent 174 and force the retaining clip detent 174 downward as the frame 130 pass past the retaining clip detent 174 and into the frame channel 152.

The retaining clip 170 may further include a lever or button 176 at its proximal end. Similar to the retaining clip detent 174, the lever or button 176 may extend upwardly from the longitudinal member 172. In various embodiments, one or more springs may bias the retaining clip 170 toward the retaining or locked position. Pressing the lever or button 176 with sufficient force to overcome the spring bias may cause the longitudinal member 172 to pivot about its coupling to the carriage body 150 toward an opened or unlocked position. In particular, such pivoting may move the retaining clip detent 174 downward, thus retracting the retaining clip detent 174 from the frame channel 152 and permitting withdrawal of the frame 130 from the frame channel 152.

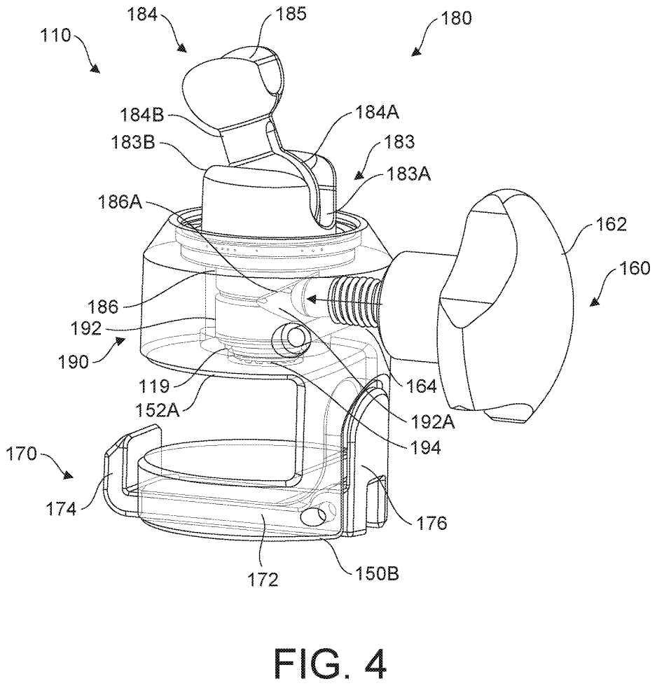

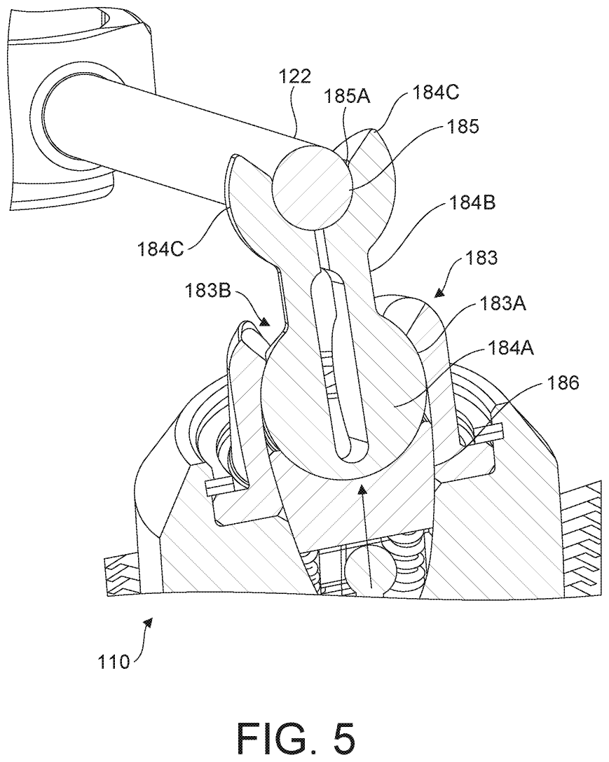

As shown in FIGS. 4 and 5, the arm clamp 180 may be positioned along a top side of the carriage body 150 and may comprise a cup 183, a ball stud 184, and an arm clamp piston 186. The cup 183 may comprise an inner surface 183A that narrows, tapers, or is otherwise angled toward an upper opening 183B in the cup 183. In various embodiments, the diameter of the upper opening 183B is smaller than the diameter of a ball 184A of the ball stud 184. In some embodiments, the cup 183 may be continuous with or otherwise integrated with the top side of the carriage body 150.

In some embodiments, the cup 183 may be formed in various ways and in various shapes. While the present disclosure contemplates the cup 183 as comprising a spherical upper surface and a cylindrical lateral surfaces that provide the cup 183 with a circular cross section, the cup 183 may have be various shapes. For example, the cup 183 may be implemented with lateral surfaces having other cross-sections such as, for example, elliptical cross sections, rectangular cross sections, hexagonal cross sections, etc.

The ball stud 184 may comprise a ball 184A, a shaft 184B with a proximal end coupled to the ball 184A, and clasps 184C coupled to a distal end of the shaft 184B. In particular, the ball 184A and shaft 184B may be bifurcated into two or more branches with each branch comprising a clasp 184C at the distal end. The ball 184A of the ball stud 184 may be housed or otherwise positioned within the cup 183 such that external surfaces of the ball 184A engage the inner surface 183A of the cup 183 and the shaft 184B extends through the opening 183B of the cup 183.

The clasps 184C may define a passage 185 sized to receive and partial circumscribe or surround one or more external surfaces of a longitudinal arm member 122 of the retractor arm 120. In various embodiments, the bifurcation of the ball stud 184 imparts the passage 185 with elasticity. Namely, the branches of the shaft 184B effectively operate as leaf springs that bias the clasps 184C toward an unlocked position in which the passage 185 and a lateral opening 185A to such passage 185 is sufficiently large to (i) permit a longitudinal arm member 122 of the retractor arm 120 to enter the passage 185 and be received by the clasps 184C; and (ii) permit a longitudinal arm member 122 of the retractor arm 120 to be withdrawn from the clasps 184C.

Furthermore, the bifurcation of the ball stud 184 may enable the inner surface 183A to contract the ball 184A. Such contraction of the ball 184A may cause contraction of the shaft 184B and contraction of the clasps 184C coupled to the distal ends of the shaft 184B toward a locked or tightened position. When in the locked position, the passage 185 through the clasps 184C and the lateral opening 185A to the passage 185 may be sufficient reduced or contracted to prevent withdrawal of a longitudinal arm member 122 of the retractor arm 120 from the clasps 184C.

The arm clamp piston 186 may be positioned in the carriage body 150 below the ball 184A of the ball stud 184. In particular, one or more inner surfaces of the carriage body 150 may engage one or more outer surfaces of the arm clamp piston 186 so as to restrict movement of the arm clamp piston 186. In particular, the carriage body 150 may permit the piston to move upward and downward within the carriage body 150 while restricting radial and/or lateral movement of the arm clamp piston 186.

As shown, the arm clamp piston 186 may further include an inclined, ramped, or otherwise angled surface 186A radially aligned so as to receive the distal end of the knob 160. In particular, the inclined surface 186A is angled upward in a radially outward direction. Due to such configuration, as the knob 160 is rotated toward a tightening direction, the distal end of the knob 160 advances inwardly upon the inclined surface 186A and forces the arm clamp piston 186 in an upward direction. Such upward movement of the arm clamp piston 186 forces the ball 184A against the inner surface 183A of the cup 183, thus causing the ball 184A to contract. Moreover, such contraction of the ball 184A causes contraction of the shaft 184B and the clasps 184C coupled thereto. Accordingly, by sufficiently tightening of the knob 160, a longitudinal arm member 122 of the retractor arm 120 may be locked or otherwise secured within the clasps 184C.

As further shown in FIGS. 4 and 5, the frame clamp 190 may comprise a frame clamp piston 192 positioned within the carriage body 150 below the arm clamp piston 186 of the arm clamp 180. Similar to the arm clamp piston 186, one or more inner surfaces of the carriage body 150 may engage one or more outer surfaces of the frame clamp piston 192 so as to restrict movement of the frame clamp piston 192. In particular, the carriage body 150 may permit the frame clamp piston 192 to move upward and downward within the carriage body 150 while restricting radial and/or lateral movement of the frame clamp piston 192.

As shown, the frame clamp piston 192 may further include an inclined, ramped, or otherwise angled surface 192A that is radially aligned with the knob 160 so as to receive the distal end of the knob 160. In particular, the inclined surface 192A may be angled downward in a radially outward direction. Due to such configuration, as the knob 160 is rotated toward a tightening direction, the distal end of the threaded shaft 164 may advance inwardly upon the inclined surface 192A and force the frame clamp piston 192 in a downward direction.

As further shown, the frame clamp 190 may include a frame clamp detent 194 along a bottom side of the frame clamp piston 192. The frame clamp detent 194 may protrude from the bottom side of the frame clamp piston and through an opening 119 in the top surface 152A of the frame channel 152 and into the frame channel 152. Due to such configuration, downward movement of the frame clamp piston 192 may cause the frame clamp detent 194 to apply a retaining force against a top surface of the frame 130. Thus, with sufficient tightening of the knob 160, the frame clamp 190 may prevent and/or restrict removal of the frame 130 from the frame channel 152 and/or may prevent and/or restrict movement of the carriage 110 along the frame 130.

In some examples, a surface 194A of the frame clamp detent 194 may comprise a grooved, rough, or smooth surface. In some examples, the surface 194A may be grooved in order to increase friction, and therefore resistance between the frame clamp detent 194 and the frame 130. Such increased resistance may increase stability and may aid in retaining the carriage 110 in a tightened configuration relative to the frame 130.

The present disclosure includes reference to certain examples; however, it will be understood by those skilled in the art that various changes may be made and equivalents may be substituted without departing from the scope of the disclosure. In addition, modifications may be made to the disclosed examples without departing from the scope of the present disclosure. Therefore, it is intended that the present disclosure not be limited to the examples disclosed, but that the disclosure will include all examples falling within the scope of the appended claims.

What is claimed is:

1. A surgical retractor system, comprising:
a frame,
a carriage coupled to the frame, the carriage comprising:
a knob, and
a ball stud comprising a ball at a proximal end and clasps at a distal end; and
a retractor arm coupled to the frame via the clasps of the carriage;
wherein the retractor arm is at least partially surrounded by the clasps of the ball stud;
wherein rotation of the knob in a first direction tightens the clasps around the retractor arm; and
wherein the carriage is movable along the frame to position the retractor arm.

2. The surgical retractor system of claim 1, wherein:
the carriage comprises a retaining clip; and
the retaining clip prevents removal of the frame from the carriage when in a retaining position.

3. The surgical retractor system of claim 1, wherein:
the carriage comprises a cup; and
an inner surface of the cup engages the ball of the ball stud.

4. The surgical retractor system of claim 1, wherein:
the carriage comprises a frame clamp piston with an inclined surface radially aligned with the knob; and
rotation of the knob causes a distal end of the knob to engage the inclined surface and force the frame clamp piston toward the frame.

5. The surgical retractor system of claim 4, wherein
the carriage comprises an arm clamp piston positioned above the frame clamp piston;
the arm clamp piston comprises an inclined surface radially aligned with the knob; and
rotation of the knob causes a distal end of the knob to engage the inclined surface of the arm clamp piston and cause the arm clamp piston to contract the ball of the ball stud against an inner surface of the carriage.

6. The surgical retractor system of claim 1, wherein
the carriage comprises an arm clamp piston with an inclined surface radially aligned with the knob; and
rotation of the knob causes a distal end of the knob to engage the inclined surface and cause the arm clamp piston to contractt the ball of the ball stud against an inner surface of the carriage.

7. The surgical retractor system of claim 6, wherein contraction of the ball causes contraction of the clasps around the retractor arm.

8. A surgical retractor system, comprising:
a carriage body comprising a top side, a bottom side, and one or more lateral sides between the top side and the bottom side;
an arm clamp coupled to the carriage body and configured to secure a retractor arm;
a frame channel that passes laterally through the carriage body;
a frame that passes laterally through the carriage body via the frame channel;
a retaining clip comprising a detent that prevents removal of the frame from the frame channel when in a retaining position;
a frame clamp piston comprising a detent that engages a surface of the frame; and
a knob comprising a thread shaft through a lateral side of the one or more lateral sides; and
wherein tightening of the knob forces the clamp piston toward the surface of the frame.

9. The surgical retractor system of claim 8, wherein:
the retaining clip is positioned along a bottom side of the carriage body; and
the detent of the retaining clip extends upwardly from a distal end of the retaining clip and above a bottom surface of the frame channel.

10. The surgical retractor system of claim 9, wherein
the retaining clip comprises a button extending from a proximal portion of the retaining clip;
the proximal portion of the retaining clip is coupled toward a back side of the carriage body; and
pressing the button causes the retaining clip to pivot the detent of the retaining clip away from the frame channel.

11. The surgical retractor system of claim 8, wherein tightening of the knob causes the arm clamp to tighten around the retractor arm.

12. The surgical retractor system of claim 8, comprising:
an arm clamp piston positioned above the frame clamp piston;
wherein the arm clamp piston comprises an inclined surface radially aligned with the knob; and
wherein tightening of the knob causes a distal end of the knob to engage the inclined surface of the arm clamp piston and cause the arm clamp piston to tighten the arm clamp around the retractor arm.

13. The surgical retractor system of claim 8, comprising:
an arm clamp coupled to the carriage body;
wherein the arm clamp comprises a cup and a ball stud;
wherein the ball stud comprises a ball in the cup and clasps that extend beyond the cup; and
wherein the clasps are configured to secure a retractor arm to the frame via the ball stud.

14. The surgical retractor system of claim 13, comprising:
an arm clamp piston with an inclined surface radially aligned with the knob; and
wherein tightening of the knob causes a distal end of the knob to engage the inclined surface and cause the arm clamp piston to contract the ball of the ball stud against an inner surface of the cup.

15. The surgical retractor system of claim 14, wherein contraction of the ball causes contraction of the clasps around the retractor arm.

16. A carriage for securing a retractor arm to a frame, the carriage comprising:
a carriage body comprising a top side, a bottom side, and one or more lateral sides between the top side and the bottom side;
an arm clamp coupled to the carriage body, wherein the arm clamp is configured to secure the retractor arm to the carriage body;
a frame channel that passes laterally through the carriage body;
a retaining clip comprising a detent that at least partially blocks an opening to the frame channel when in a retaining position;
a frame clamp piston comprising a detent that extends into the frame channel; and
a knob comprising a thread shaft through a lateral side of the one or more lateral sides; and
wherein tightening of the knob forces the frame clamp piston toward the frame channel.

17. The carriage of claim 16, wherein:
the retaining clip is positioned along a bottom side of the carriage body; and
the detent of the retaining clip extends upwardly from a distal end of the retaining clip and above a bottom surface of the frame channel.

18. The carriage of claim 16, wherein tightening of the knob causes the arm clamp to tighten around the retractor arm.

19. The carriage of claim 16, comprising:
an arm clamp piston positioned above the frame clamp piston;
wherein the arm clamp piston comprises an inclined surface; and
wherein tightening of the knob causes a distal end of the knob to engage the inclined surface of the arm clamp piston and cause the arm clamp piston to tighten the arm clamp around the retractor arm.

20. The carriage of claim 16, wherein:
the arm clamp comprises a cup and a ball stud;
the ball stud comprises a ball in the cup and clasps that extend beyond the cup; and
the clasps are configured to secure the retractor arm to the carriage body via the ball stud.
